(19) United States
(12) Patent Application Publication
(10) Pub. No.: US 2023/0109015 A1
(43) Pub. Date: Apr. 6, 2023

(54) SURGICAL IMPACTOR NAVIGATION SYSTEMS AND METHODS

(71) Applicant: 360 Knee Systems Pty Ltd, New South Wales (AU)

(72) Inventors: Brad Miles, New South Wales (AU); Joshua Twiggs, New South Wales (AU); Willy Theodore, New South Wales (AU)

(73) Assignee: 360 Knee Systems Pty Ltd, New South Wales (AU)

(21) Appl. No.: 17/905,683

(22) PCT Filed: Feb. 26, 2021
(86) PCT No.: PCT/AU2021/050174

§ 371 Date: Sep. 6, 2022

(30) Foreign Application Priority Data
Mar. 4, 2020 (AU) 2020900655

(51) Int. Cl. A61B 34/10; A61F 2/46; A61F 2/36; G06T 7/77; G16H 40/63; G16H 20/40; G16H 30/40

(52) U.S. Cl. CPC A61B 34/10; A61F 2/4607; A61F 2/36; G06T 7/77; G16H 40/63; G16H 20/40; G16H 30/40; A61B 2034/2055

(57) ABSTRACT

This disclosure relates to systems for assisting surgeons in implanting joint replacement implant components. One aspect provides a system for assisting a surgeon in implanting a joint replacement implant component during a surgery of replacing a joint. The system comprises: an instrument for medullary canal preparation; a video camera to capture image data of the instrument; a computer system to: store a surgical plan; determine a pose of the instrument relative to the bone or the joint based on the image data from the video camera; assess the pose of the instrument against the surgical plan; and provide an indication to the surgeon of a clinical consequence of the pose in relation to the surgical plan.

### CROSS-REFERENCE TO RELATED APPLICATIONS

The present application claims priority from Australian Provisional Patent Application No 2020900655 filed on 4 Mar. 2020, the contents of which are incorporated herein by reference in their entirety.

### TECHNICAL FIELD

This disclosure relates to systems and methods for assisting a surgeon in implanting a joint replacement implant component during a surgery of replacing a joint.

### BACKGROUND

Many orthopaedic surgeries include the use of a broaching instrument for medullary canal preparation. In particular, a surgeon may insert an implant component, such as a femoral component of a hip joint replacement, into a medullary canal of a bone of the joint. For example, a surgeon may insert a femoral component into the medullary canal of the femur, by hammering a broach into the medullary canal. The broach creates a void in the femur, into which the surgeon then inserts the implant component.

It is important that the broach is inserted at a pose that allows for a satisfactory outcome of the surgery. This means, a small deviation from the optimum pose by about 5 degrees or less, may have a negative impact on the patient outcome. It is difficult, however, for a surgeon to consistently control the pose of the broach such that it enters the bone optimally.

### SUMMARY

Disclosed herein are systems and methods that use video capture and machine vision to identify a broaching instrument in the video data and calculate the pose of the broaching instrument. This can then be matched against a surgical plan and the result is provided to the surgeon. This way, the surgeon has real-time feedback on where the broach is heading and can make adjustments while the surgeon inserts the broach into the bone.

There is described a system for assisting a surgeon in implanting a joint replacement implant component during a surgery of replacing a joint. The system may comprise:

an instrument for medullary canal preparation;

a video camera to capture image data of the instrument;

a computer system to:
store a surgical plan;
determine a pose of the instrument relative to the bone or the joint based on the image data from the video camera;
assess the pose of the instrument against the surgical plan; and
provide an indication to the surgeon of a clinical consequence of the pose in relation to the surgical plan.

The surgical plan may comprise a two-dimensional plan.

The computer system may be configured to create a three-dimensional surgical plan from two or more two-dimensional medical images.

The medical images may be X-ray images.

The surgical plan may comprises a three-dimensional surgical plan.

The instrument may be one of a broaching instrument and a rasping instrument.

The instrument may comprise a broach handle with an impact surface to receive impact from a surgeon operated hammer.

The instrument may be an automatic impactor that generates impact energy and delivers the impact energy to a broach for medullary canal preparation.

The automatic impactor may be controlled.

The impactor may deliver a predefined amount of energy to the broach.

The clinical consequence may comprise a risk stratification.

The clinical consequence may comprise a simulated performance metric determined by simulating a three-dimensional model of the joint based on the pose of the broaching instrument.

Simulating the three-dimensional model may be based on an implant placement defined by the pose of the broaching instrument.

The computer system may be configured to generate a graphical display of the joint and an indication of the pose of the broaching instrument in relation to the joint.

The computer system may be further configured to:

receive an x-ray image;

display the x-ray image; and

overlay over the x-ray image an indication of the pose of the broaching instrument.

Determining the pose of the instrument may comprise detecting objects in the image data and fitting an object model to the objects.

A two-dimensional marker may be affixed to the instrument.

Determining the pose of the instrument may comprise determining the pose of the two-dimensional marker.

There is also described a method for assisting a surgeon in implanting a joint replacement implant component during a surgery of replacing a joint. The method may comprise:

storing a surgical plan;

determining a pose of the instrument relative to the bone or the joint based on the image data from a video camera;

assessing the pose of the instrument against the surgical plan; and

providing an indication to the surgeon of a clinical consequence of the pose in relation to the surgical plan.

The clinical consequence may comprise a risk stratification.

The clinical consequence may comprises a simulated performance metric determined by simulating a three-dimensional model of the joint based on the pose of the broaching instrument.

Simulating the three-dimensional model may be based on an implant placement defined by the pose of the broaching instrument.

determining the pose of the instrument may comprise detecting objects in the image data and fitting an object model to the objects.

A two-dimensional marker may be affixed to the instrument.

Determining the pose of the instrument may comprise determining the pose of the two-dimensional marker.

### BRIEF DESCRIPTION OF DRAWINGS

Examples of the present disclosure will now be described by way of non-limiting example only, with reference to the accompanying drawings, in which:

FIG. 1 illustrates one example of a surgical impactor navigation system for assisting surgery of a joint.

FIG. 2 illustrates another example of the surgical impactor navigation system.

FIG. 3 illustrates a process flow diagram of a method for assisting a surgeon in total joint replacement of a joint of a patient.

FIG. 4 illustrates a postoperative joint replacement X-ray.

FIG. 5 illustrates an exemplary updated digital three-dimensional model that has been manipulated to determine a postoperative range of motion of a hip joint.

FIG. 6a illustrates a schematic line drawing 600a of a patient performing a seated flexion movement.

FIG. 6b illustrates a schematic line drawing 600b of a patient performing a standing pivot extension movement.

FIG. 7 illustrates an example indication of a intraoperative simulated performance metric.

FIG. 8 illustrates another example indication of an intraoperative simulated performance metric.

FIG. 9 illustrates another example indication of an intraoperative simulated performance metric.

FIG. 10 illustrates another example indication of an intraoperative simulated performance metric.

FIG. 11 illustrates an intraoperative X-ray during a total hip replacement surgery.

FIG. 12a illustrates a perspective view of an example digital three-dimensional model with a supplemental implant component hidden.

FIG. 12b illustrates another perspective view of the digital three-dimensional model of FIG. 12a.

FIG. 12c illustrates a third perspective view of the digital three-dimensional model of FIG. 12a.

FIG. 13 illustrates an example scenario of medullary canal preparation of a femur.

### DESCRIPTION OF EMBODIMENTS

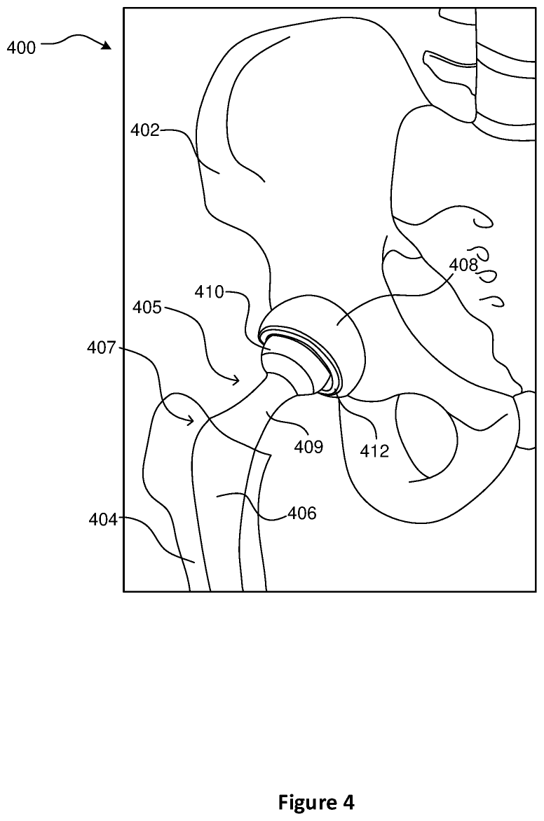

Surgical impactor navigation systems and methods for assisting with surgery are described. Surgeries, such as joint replacement surgeries have many parameters that can be influenced by the surgeon. For example, FIG. 4 illustrates a postoperative joint replacement X-ray 400. An implant component assembly 405 is used in the joint replacement. The implant component assembly 405 comprises one or more implant components. In some examples, the implant component assembly 405 comprises an implant component 406. The implant component assembly 405 also includes a number of supplemental implant components 407.

The postoperative joint replacement X-ray 400 of FIG. 4 is of a hip joint of a patient after total hip replacement. FIG. 4 shows the patient's pelvis 402, femur 404 and the implant component assembly 405. The implant component assembly 405 comprises the implant component 406 in the form of femoral stem 406. The implant component assembly 405 also comprises a plurality of supplemental implant components 407. The supplemental implant components 407 comprise an acetabular component 408, a neck 409, an implanted femoral head 410 and a liner 412.

During total hip replacement surgery, the surgeon removes the patient's femoral head, reams the patient's natural acetabulum with a reamer, and implants the the acetabular component 408 in the resulting recess. The acetabular component 408 is a hollow hemi-spherical component. The surgeon then implants supplemental implant components 407. The liner 412 is received by the acetabular component 408. The liner 412 is a hollow hemi-spherical component. The liner 412 is often polymeric. The surgeon implants the femoral stem 406 in the patient's femur (such as by hammering a broach into the medullary canal), and connects the neck 409 to the femoral stem 408. The surgeon connects the implanted femoral head 410 to the neck 408. The femoral stem 408 is an elongate component. The neck 409 is an elongate component. The implanted femoral head 410 is a generally spherical component. The acetabular component 408 and liner 412 receive the implanted femoral head 410. The acetabular component 408, liner 412, femoral stem 408, neck 409 and implanted femoral head 410 cooperate to emulate the mechanics of a natural hip joint.

Surgeries such as total hip replacements have many parameters that the surgeon can modify. For example, in the context of the illustrated total hip replacement, the surgeon can modify leg length, horizontal centre of rotation, vertical centre of rotation, acetabular inclination, acetabular anteversion, femoral stem positioning and cement mantle thickness. In some examples, these parameters may be measured as described in Vanrusselt, Jan & Vansevenant, Milan & Vanderschueren, Geert & Vanhoenacker, Filip. (2015). “Postoperative radiograph of the hip arthroplasty: what the radiologist should know”, the contents of which is incorporated herein by reference. The disclosed surgical impactor navigation systems and methods can assist with joint surgery.

### System Overview

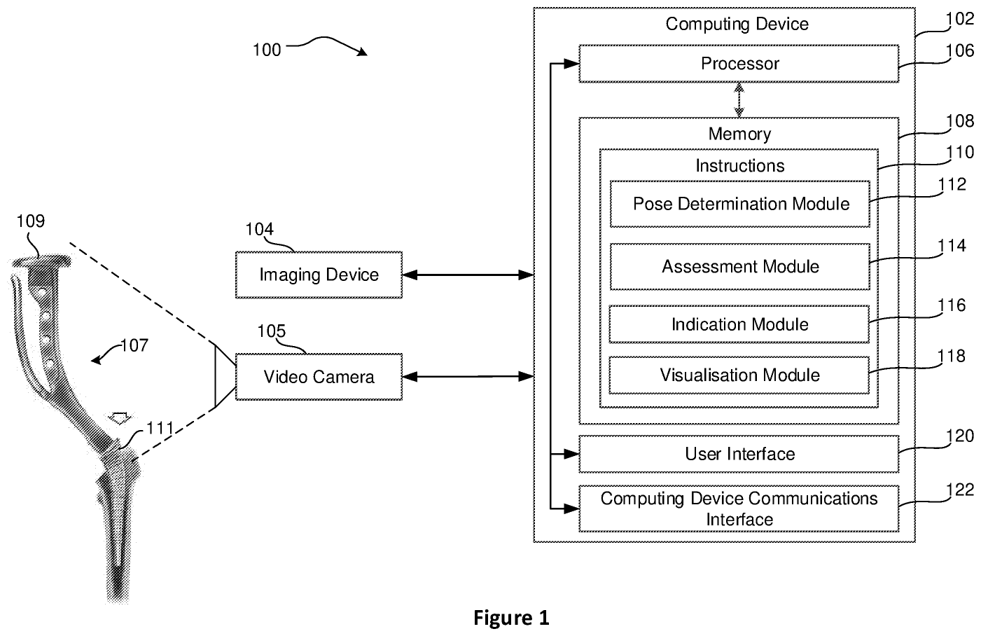

FIG. 1 illustrates an surgical impactor navigation system 100 for assisting surgery of a joint. The system 100 comprises a computing device 102. The computing device 102 comprises a processor 106 and a memory 108. The system 100 also comprises an imaging device 104, such as an X-ray device. The imaging device 104 is in communication with the computing device 102. System 100 further comprises a video camera 105, which captures image data of a surgical instrument 107 for medullary canal preparation. As shown in FIG. 1, the instrument 107 may be a broach handle or a handle of a rasping instrument. Broach handle 107 comprises an impact surface 109. The surgeon hits the impact surface 109 with a hammer to deliver impact energy to a broach 111 at the end of the broach handle 107. The impact energy drives the broach 111 into the medullary canal of the bone.

The surgical instrument 107 may also comprise a femoral rasp instead of broach 111. In yet a further example, the surgical instrument 107 may comprise an automatic impactor, which delivers a controlled amount of impact to the broach 111 or rasp. One example of an automatic impactor is the KINCISE™ Surgical Automated System by Johnson & Johnson Medical Devices.

It is also noted that system 100 may comprise further cameras to capture image data of the surgical instrument 107 from different viewpoints to facilitate 3D imaging. For example, video camera 105 may be part of a three-dimensional stereo vision system.

The processor 106 is configured to execute instructions 110 stored in memory 108 to cause the system 100 to function according to the described methods. The instructions 110 may be in the form of program code. The processor 106 may comprise one or more microprocessors, central processing units (CPUs), application specific instruction set processors (ASIPs), application specific integrated circuits (ASICs) or other processors capable of reading and executing instruction code.

Memory 108 may comprise one or more volatile or non-volatile memory types. For example, memory 108 may be a non-transitory compute readable medium, such as a hard drive, a solid state disk or CD-ROM. Memory 108 may comprise one or more of random access memory (RAM), read-only memory (ROM), electrically erasable programmable read-only memory (EEPROM) or flash memory. Memory 108 is configured to store program code accessible by the processor 106. The program code comprises executable program code modules. In other words, memory 108 is configured to store executable code modules configured to be executable by the processor 106. The executable code modules, when executed by the processor 106 cause the system 100 to perform the methods disclosed herein.

The computing device 102 may also comprise a user interface 120. The user interface 120 is configured to receive one or more inputs from a user. The user interface 120 is also configured to provide one or more outputs to the user. In some examples, the user can submit a request to the computing device 102 via the user interface 120, and the computing device 102 can provide an output to the user via the user interface 120. The user interface 120 may comprise one or more user interface components, such as one or more of a display device, a touch screen display, a keyboard, a mouse, a camera, a microphone, buttons, switches and lights.

The computing device 102 comprises a computing device communications interface 122. The computing device communications interface 122 is configured to facilitate communication between the computing device 102, the imaging device 104 and the video camera 105. The computing device communications interface 122 may comprise a combination of communication interface hardware and communication interface software suitable for establishing, maintaining and facilitating communication over a relevant communication channel. In some examples, the computing device communications interface 122 is in the form of a computing device network interface.

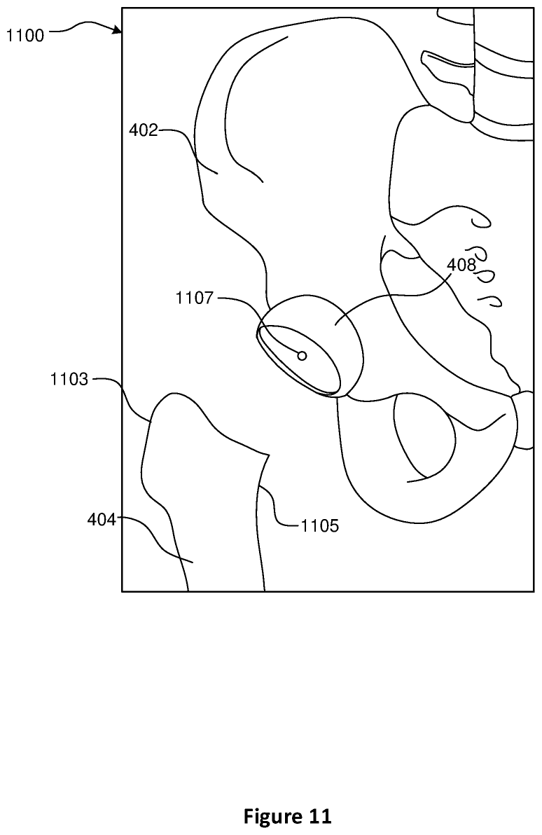

FIG. 11 illustrates an example digital X-ray image 1100. The digital X-ray image 1100 is an intraoperative X-ray image of a patient's hip. In particular, the digital X-ray image is an anterior-posterior X-ray image of the patient's hip. The digital X-ray image 1100 therefore represents an intraoperative stage of the total hip replacement surgery, with the implant component 406 (being the acetabular component) having been implanted. FIG. 11 also illustrates the patient's pelvis 1106 and the patient's femur 404.

It is to be understood that ‘image’ may refer to a two-dimensional image, such as an X-ray image stored on memory 108. In some examples, the digital X-ray image 1100 is stored in the form of a two-dimensional pixel matrix. The two-dimensional pixel matrix may comprise one intensity value for each pixel in the case of a grey scale image. Alternatively, the digital X-ray image 1100 is stored in the form of a colour model (e.g. a RGB colour model) comprising colour information of each pixel. In some examples, the colour information is carried directly by the pixel bits themselves. In some examples, the colour information is provided by a colour look-up table. The colour information may be RGB information.

However, ‘image’ may also refer to a two-dimensional projection of a three-dimensional digital model constructed from multiple two-dimensional images, such as images from a MRI or a CT scan. The surgeon can peruse this “image stack” or the two-dimensional projection on a two-dimensional screen by specifying depth values and viewing angles. Two-dimensional images and three-dimensional models may be stored on data memory 108 as multiple intensity values, such as in a two-dimensional or three-dimensional pixel matrix or as a grid model. In other examples, the two-dimensional image or the three-dimensional model is stored in a parameterised representation, such as a spline representation, and processor 106 generates a two-dimensional view on a screen (e.g. user interface 120) by interpolation based on spline parameters of the spline representation. In yet a further meaning, ‘image’ or ‘image data’ may refer to the image data generated by video camera 105, such as RGB image data from a CMOS or CCD imaging sensor.

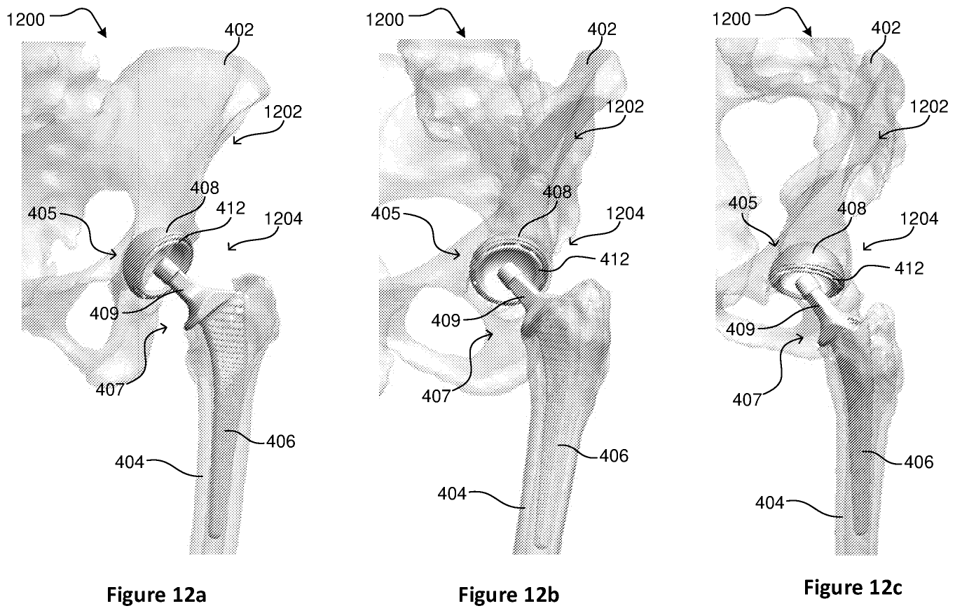

FIGS. 12a to 12c illustrate an example digital three-dimensional model 1200. In some examples the digital three-dimensional model 1200 includes details of the patient's anatomy. For example, the digital three-dimensional model 1200 can include the patient's bone and/or soft tissue structure at and around the joint that is to be replaced. The digital three-dimensional model 1200 may be a wire mesh model or finite element model. The digital three-dimensional model may represent mechanical connections for force transfer provided by the bones as well as bearing surfaces of the bones to form joints. The digital three-dimensional model 1200 can also include representation of the implant component assembly 405, including a wire mesh or finite element model of the implant component assembly 405 together with pose and 3D location and/or placement within the digital three-dimensional model 1200. As a result, the representation of the implant component assembly 405 can also represent mechanical connections for force transfer and bearing surfaces to form joints. As illustrated, one of the supplemental implant components 407 is hidden in FIGS. 12a to 12c. In particular, the implanted femoral head 410 is hidden. The digital three-dimensional model 1200 is described in more detail below.

As illustrated in FIG. 1, memory 108 comprises a pose determination module 112 configured to receive the image data from camera 105 and determine the pose of the surgical instrument 117 relative to the bone it is preparing (such as the femur) or the joint (such as the hip joint) based on the image data from the video camera 105.

It is to be understood that any receiving step may be preceded by the processor 106 determining, computing and/or storing the data that is later received. For example, the processor 106 may store the data (e.g. the digital X-ray image 1100 or image data from video camera 105) in memory 108. The processor 106 then requests the data from memory 108, such as by providing a read signal together with a memory address. The memory 108 provides the data as a voltage signal on a physical bit line and the processor 106 receives the data. It should also be understood that any receiving step may comprise the data being received from memory 108, imaging device 104, over a network via computing device communications interface 122 and/or from another device.

Memory 108 also comprises an assessment module 114 configured to assess the pose of the instrument 107 against a surgical plan. This may also comprise simulating a performance metric associated with the determined placement of the implant component 408, as will be described in more detail below.

Memory 108 also comprises an indication module 116 configured to determine an indication of a clinical consequence of the current pose in relation to the surgical plan. The clinical consequence may comprise the intraoperative simulated performance metric. In particular, the indication module 116 may be configured to provide the indication of the clinical consequence, such as the intraoperative simulated performance metric as an assessment of a current placement of the implant component 408 and/or the instrument 107, as will be described in more detail below.

Memory 108 also comprises a visualisation module 118 configured to provide the determined indication to the surgeon. In particular, the visualisation module 118 may be configured to provide the determined indication to the surgeon by way of a visual output using the user interface 120, as will be described in more detail below.

Imaging device 104 is configured to capture the digital X-ray image 1100 of the joint. Imaging device 104 may be configured to capture the digital X-ray image 1100 of the joint and the implant component 406 during the total joint replacement surgery. Furthermore, the imaging device 104 is configured to provide the captured digital X-ray image 1100 of the joint and the implant component 406 to the computing device 102. In some examples, the imaging device 104 can be an X-ray imaging device (e.g. a single-shot X-ray device or a fluoroscopy device), a computed tomography (CT) imaging device, a magnetic resonance image (MRI) imaging device, a digital camera (colour or black and white) or another type of imaging device. The advantages of using an X-ray imaging device during surgery include:

Speed: Taking the images is relatively fast.
Ease of use: The device can be easily moved into place to capture the digital X-ray image 1100, e.g., on wheels, and moved out of place after capturing the digital X-ray image 1100.
Cost: X-ray imaging devices can be cheap compared to CT or MRI imaging devices.
Low radiation exposure to the patient and the surgeon.

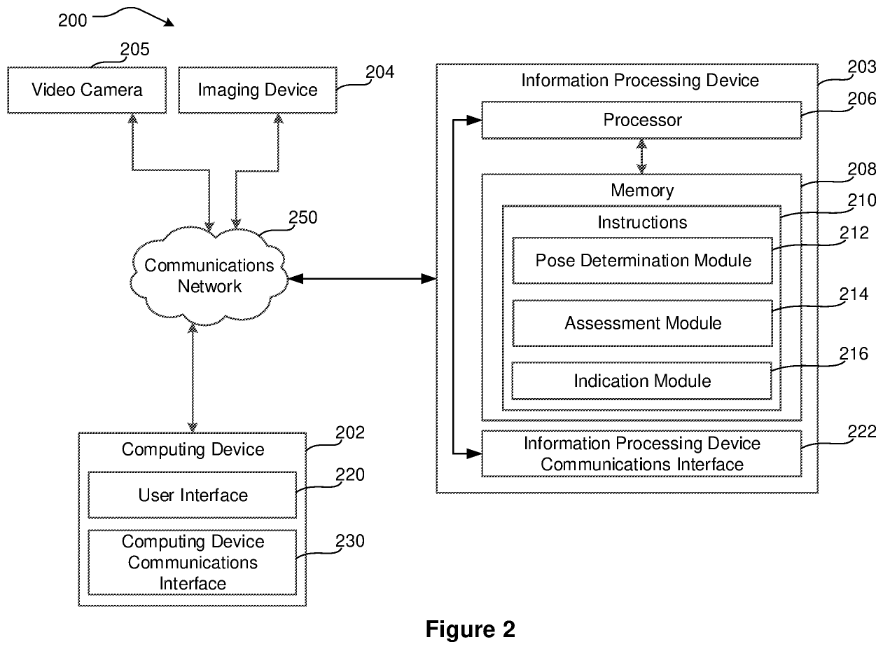

FIG. 2 illustrates another surgical impactor navigation system 200 for assisting surgery of the joint. The system 200 comprises a computing device 202. The system 200 also comprises an information processing device 203. In some examples, the computing device 202 is configured to be in communication with the information processing device 203 over a communications network 250. The system 200 also comprises an imaging device 204, such as an X-ray device. System 200 further comprises a video camera 205. The video camera 205 captures image data of a surgical instrument 207 for medullary canal preparation. The instrument 207 may be as described with reference to FIG. 1.

The imaging device 204 is configured to be in communication with the computing device 202 over the communications network 250. The imaging device 204 is configured to be in communication with the information processing device 203 over the communications network 250. The video camera 205 is configured to be in communication with the computing device 202 over the communications network 250. The video camera 205 is configured to be in communication with the information processing device 203 over the communications network 250.

Compared to FIG. 1, computing device 202 does not perform all of the data processing on the device 102 but outsources some of the processing to information processing device 203, which may be implemented as a distributed, ‘cloud’, data processing system.

The instrument 207 may be a broach handle or a handle of a rasping instrument. The broach handle 207 comprises an impact surface 209. The surgeon hits the impact surface 209 with a hammer to deliver impact energy to a broach 211 at the end of the broach handle 207. The impact energy drives the broach 211 into the medullary canal of the bone.

The surgical instrument 207 may also comprise a femoral rasp instead of broach 211. In yet a further example, the surgical instrument 207 may comprise an automatic impactor, which delivers a controlled amount of impact to the broach 211 or rasp. One example of an automatic impactor is the KINCISE™ Surgical Automated System by Johnson & Johnson Medical Devices.

It is also noted that system 200 may comprise further cameras to capture image data of the surgical instrument 207 from different viewpoints to facilitate 3D imaging. For example, video camera 205 may be part of a three-dimensional stereo vision system.

The information processing device 203 comprises a processor 206. The processor 206 is configured to execute instructions 210 stored in memory 208 to cause the system 200 to perform the methods disclosed herein. The instructions 210 may be in the form of program code. The processor 206 may comprise one or more microprocessors, central processing units (CPUs), application specific instruction set processors (ASIPs), application specific integrated circuits (ASICs) or other processors capable of reading and executing instruction code. In some embodiments, the processor 206 may be considered a first processor.

Memory 208 may comprise one or more volatile or non-volatile memory types. For example, memory 208 may be a non-transitory compute readable medium, such as a hard drive, a solid state disk or CD-ROM. Memory 208 may comprise one or more of random access memory (RAM), read-only memory (ROM), electrically erasable programmable read-only memory (EEPROM) or flash memory. Memory 208 is configured to store program code accessible by the processor 206. The program code comprises executable program code modules. In other words, memory 208 is configured to store executable code modules configured to be executable by the processor 206. The executable code modules, when executed by the processor 206 cause the system 200 to perform the methods disclosed herein. In some embodiments, the memory 208 may be considered a first memory.

The information processing device 203 comprises an information processing device communications interface 222. The information processing device 203 is configured to communicate with the imaging device 204, video camera 205 and/or the computing device 202 using the information processing device communications interface 222. The information processing device communications interface 222 may comprise a combination of communication interface hardware and communication interface software suitable for establishing, maintaining and facilitating communication over a relevant communication channel. In some examples, the information processing device communications interface 222 is in the form of an information processing device network interface. Examples of a suitable communications network 250 include a cloud server network, wired or wireless internet connection, Bluetooth^{R} or other near field radio communication, and/or physical media such as USB. The processor 206 may receive data via the information processing device communications interface 222 and/or from memory 208.

As illustrated in FIG. 2, memory 208 comprises a pose determination module 212 configured to receive the image data from video camera 205 and determine the pose of the surgical instrument 117 relative to the bone it is preparing (such as the femur) or the joint (such as the hip joint) based on the image data from the video camera 250.

In some examples, the digital X-ray image 1100 is a two-dimensional image. In particular, the digital X-ray image 1100 may be an X-ray image or a fluoroscopy image. The digital X-ray image 1100 may be captured by the imaging device 204.

It is to be understood that any receiving step may be preceded by the processor 106 determining, computing and/or storing the data that is later received. For example, the processor 206 may store the data (e.g. the digital X-ray image 1100) in memory 208. The processor 206 then requests the data from memory 208, such as by providing a read signal together with a memory address. The memory 208 provides the data as a voltage signal on a physical bit line and the processor 206 receives the data. It should also be understood that any receiving step may comprise the data being received from memory 208, computing device 202, information processing device 203, imaging device 204, over the communications network 250 via computing device communications interface 222 and/or from another device.

Memory 208 also comprises an assessment module 214 configured to assess the pose of the instrument 107 against a surgical plan. This may also comprise simulating a performance metric associated with the determined placement of the implant component 406, as will be described in more detail below.

Memory 208 also comprises an indication module 216 configured to determine an indication of the clinical consequence of the current pose in relation to the surgical plan. The clinical consequence may comprise the intraoperative simulated performance metric. In particular, the indication module 216 may be configured to provide the indication of the clinical consequence, such as the intraoperative simulated performance metric as an assessment of a current placement of the implant component 408 and/or the instrument 207, as will be described in more detail below.

The computing device 202 comprises the computing device communications interface 230 and the user interface 220. The computing device 202 is configured to communicate with the information processing device 203, video camera 205 and/or the imaging device 204 over the communications network 250 using the computing device communications interface 230. The computing device communications interface 230 may comprise a combination of communication interface hardware and communication interface software suitable for establishing, maintaining and facilitating communication over a relevant communication channel. In some embodiments, the computing device 202 comprises a computing device processor. The computing device processor may be considered a second processor. In some embodiments, the computing device 202 comprises a computing device memory. In some embodiments, the computing device memory may be considered a second memory. The computing device memory may store program code accessible by the computing device processor. The program code may be configured to cause the computing device processor to perform the functionality described herein.

The user interface 220 is configured to receive one or more inputs from a user. The user interface 220 is also configured to provide one or more outputs to the user. In some examples, the user can submit a request to the computing device 202 via the user interface 220, and the computing device 202 can provide an output to the user via the user interface 220. In some examples, the user interface 220 is configured to provide the indication determined by the indication module 216 by way of a visual output. The user interface 220 may comprise one or more user interface components, such as one or more of a display device, a touch screen display, a keyboard, a mouse, a camera, a microphone, buttons, switches and lights.

Imaging device 204 is configured to capture the digital X-ray image 1100 of the joint and the implant component 406 during the total joint replacement surgery. Furthermore, the imaging device 204 is configured to provide the captured digital X-ray image 1100 of the joint and the implant component 406 to the information processing device 203 and/or the computing device 202. In the illustrated example, the imaging device 204 is configured to transmit the digital X-ray image 1100 of the joint and the implant component 406 to the information processing device 203 and/or the computing device 202 using the communications network 220. In some examples, the imaging device 104 can be a X-ray imaging device (e.g. a single-shot X-ray device or a fluoroscopy device), a computed tomography (CT) imaging device, a magnetic resonance image (MRI) imaging device, a digital camera (colour or black and white) or another type of imaging device as previously described.

### Exemplary Embodiment

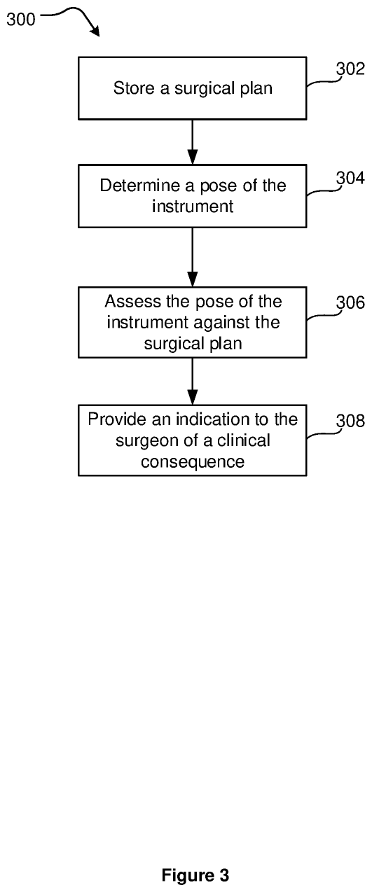

FIG. 3 illustrates a process flow diagram of a computer-implemented method 300 for assisting surgery of a joint, according to some examples. In some examples, the method 300 is performed by the surgical impactor navigation system 100, as will be described in more detail below. In some examples, the method 300 is performed by the surgical impactor navigation system 200, as will be described in more detail below.

FIG. 3 is to be understood as a blueprint for a software program and may be implemented step-by-step, such that each step in FIG. 3 may, for example, be represented by a function in a programming language, such as C++ or Java. The resulting source code is then compiled and stored as computer executable instructions 110, 210 on memory 108 in the case of system 100, and on memory 208 in the case of system 200.

Prior to commencing total joint replacement surgery, it can be beneficial to determine a surgical plan. In some examples, the digital three-dimensional model 1200 can represent the surgical plan. A surgeon can adjust implant component sizing and pose relative to the patient's anatomy in the digital three-dimensional model 1200, and use the model as a baseline to monitor intraoperative surgical progress.

### Method 300 Performed by System 100

In some examples, the computing device 102 generates the digital three-dimensional model 1200. In some examples, another computing device generates the digital three-dimensional model 1200. The digital three-dimensional model 1200 is a digital model. The digital three-dimensional model 1200 may be a hip, knee, shoulder, elbow or another joint. The digital three-dimensional model 1200 comprises an anatomical three-dimensional model 1202. The anatomical three-dimensional model 1202 is a three-dimensional model of the patient's anatomy. In particular, the anatomical three-dimensional model 1202 is a three-dimensional model of the joint to be replaced in the joint replacement surgery. In some examples, the anatomical three-dimensional model 1202 is a three-dimensional model of the patient's pre-operative anatomy. The anatomical three-dimensional model 1202 may be modified to represent the patient's anatomy after the surgery (their postoperative anatomy). For example, in cases where the patient's bone is to be cut during the surgery, the cut(s) can be included in the representation of the bone in the anatomical three-dimensional model 1202. In some examples, the anatomical three-dimensional model 1202 includes both a pre-operative anatomical three-dimensional model and postoperative anatomical three-dimensional model. In said examples, the user of the system 100 may be able to toggle between the pre-operative anatomical three-dimensional model and postoperative anatomical three-dimensional model.

Computing device 102 (or a different computing device) generates the digital three-dimensional model 1200 using information provided by a preoperative imaging device. The preoperative imaging device can be a CT imaging device or an MRI imaging device, for example.

In some examples, the preoperative imaging device is configured to provide the information to the processor 106. The processor 106 processes the information provided by the preoperative imaging device to generate the anatomical three-dimensional model 1202. The anatomical three-dimensional model 1202 is then stored in memory 108.

In some examples, a model generating computing device (not shown) processes the information provided by the preoperative imaging device to generate the anatomical three-dimensional model 1202. In said examples, the anatomical three-dimensional model 1202 is provided to the computing device 102. The anatomical three-dimensional model 1202 is then stored in memory 108.

In some examples, the digital three-dimensional model 1200 also comprises an implant component assembly three-dimensional model 1204. The implant component assembly three-dimensional model 1204 is a digital model. The implant component assembly three-dimensional model 1204 is a three-dimensional representation of the implant component assembly 405. For example, the implant component assembly three-dimensional model 1204 can comprise three-dimensional models of the implant component 406, and the one or more supplemental implant components 407. The implant component 406 can be in the form of the femoral stem 408 as previously described. The supplemental implant components 407 can be in the form of the acetabular component 408, neck 409, implanted femoral head 410 and liner 412 as previously described.

In some examples, the digital three-dimensional model 1200 represents the intended joint configuration after the surgery. That is, the implant component assembly three-dimensional model 1204 is positioned with respect to the anatomical three-dimensional model 1202 such that the digital three-dimensional model 1200 represents the intended joint configuration after the surgery. In that respect, the digital three-dimensional model 1200 can be considered a surgical plan.

The digital three-dimensional model 1200 can be transformed, such as rotated, translated and/or scaled, to correspond with the actual sizing of the patient's anatomy and the implant component assembly 405. That is, a measurement between a first point and a second point of the anatomical three-dimensional model 1202 and/or the implant component assembly three-dimensional model 1204 can be the same as a measurement between a corresponding first point and a corresponding second point of the patient's anatomy and/or the implant component assembly 405.

The implant component assembly 405, and therefore, each implant component 406 and/or each supplemental implant component 407 can be provided in a plurality of sizes. For example, each of the acetabular component 408, liner 412, femoral stem 406, neck 409 and/or femoral head 410 used in the total hip replacement illustrated in FIG. 4 can be provided in a plurality of sizes. The size of each implant component 406 and/or each supplemental implant component 407 can be determined in the digital three-dimensional model 1200.

In some examples of the digital three-dimensional model 1200, the pose of each implant component 406 and/or each supplemental implant component 407 and/or the implant component assembly 405 is determined manually. That is, a user of the system 100 can observe the patient's anatomy and/or the anatomical three-dimensional model 1202, and select a pose for each implant component 406 and/or each supplemental implant component 407 in the digital three-dimensional model 1200.

In some examples, the computing device 102 automatically determines the size of each implant component 406, each supplemental implant component 407 and/or the implant component assembly 406 of the digital three-dimensional model 1200. The determined size of each implant component 406 and/or each supplemental implant component 407 may be optimized based on anatomical geometry of the patient.

In some examples, the computing device 102 automatically determines the pose of each implant component 406 and/or each supplemental implant component 407. The determined pose of each implant component 406 and/or each supplemental implant component 407 may be optimized based on anatomical geometry of the patient.

When in the context of the total hip replacement, the digital three-dimensional model 1200 can include the patient's pelvis 406 and femur 404. The implant components used in the total hip replacement, as illustrated in FIG. 4, comprise the acetabular component 408, the liner 412, the femoral stem 408, neck 409 and the implant femoral head 410. Thus, the implant component assembly three-dimensional model 1204 for the total hip replacement can include three-dimensional representations of the acetabular component 408, liner 412, the femoral stem 408, neck 409 and/or the implant femoral head 410 to be used in the surgery.

In some examples, the computing device 102 processes the digital three-dimensional model 1200. In some examples, the model generating computing device, or another computing device processes the digital three-dimensional model 1200 and transmits the processed digital three-dimensional model 1200 to the computing device 102.

Processing the digital three-dimensional model 1200 may comprise determining one or more digital three-dimensional model parameters. The digital three-dimensional model parameters may comprise locations of one or more three-dimensional model landmarks. The three-dimensional model landmarks may be, in the case of a total hip replacement, the patient's greater trochanter 1103, lesser trochanter 1107, femoral stem alignment, femoral shaft alignment and/or the centre of rotation of the implanted femoral head 1107. The three-dimensional landmarks may comprise a number of pelvic landmarks, for example, the anterior superior iliac spine, anterior inferior iliac spine, pubic symphysis, obturator foramen, acetabular floor, sacrum, coccyx and/or greater sciatic notch. The three-dimensional landmarks may comprise a number of femoral landmarks, for example, the piriformis fossa and/or intertrochanteric ridge. Each three-dimensional model landmark may have an associated landmark location. Each landmark location may be a Cartesian coordinate in the reference frame of the digital three-dimensional model 1200.

The one or more three-dimensional model parameters may comprise one or more three-dimensional model measurements. The three-dimensional model measurements are indicative of a distance between two or more three-dimensional model landmarks. The three-dimensional model measurements may be, for example, leg length, acetabular inclination, acetabular anteversion and/or cement mantle thickness, femoral offset, anterior offset, stem varus/valgus angle, and/or the distance between one or more of the landmarks previously described.

Although the digital three-dimensional model is described as being processed after generation, in some examples, each of the anatomical three-dimensional model 1202 and/or the implant component assembly three-dimensional model 1204 are processed before being used to generate the digital three-dimensional model 1200. Thus, the digital three-dimensional model parameters may comprise anatomical three-dimensional model parameters. The anatomical three-dimensional model parameters may be determined from the anatomical three-dimensional model 1202. Furthermore, the digital three-dimensional model parameters may comprise implant component assembly three-dimensional model parameters. The implant component assembly three-dimensional model parameters may be determined from the implant component assembly three-dimensional model 1204.

The computing device 102 stores the surgical plan in memory 108. That is, the computing device 102 stores the processed digital three-dimensional model 1200 in memory 108. The computing device 102 stores the digital three-dimensional model 1200, and the associated digital three-dimensional model parameters.

The surgical plan comprises information about the planned position (location and pose) of the one or more implant components that are to be implanted into the patient to replace the joint. As previously described, the surgical plan is created preoperatively and may be based on medical imaging data, such as preoperative X-ray images, CT scans or others. In one example, the surgical plan is a two-dimensional plan similar to a two-dimensional map of one or more bones and implants located in relation to the bones. As such, the implant locations may be defined by two coordinates (x and y) and the pose of the implant may be defined by one angle. In that case, the surgical plan may be based on a single two-dimensional preoperative image, such as a single X-ray image.

In another example, processor 106 receives multiple two-dimensional images and creates a three-dimensional surgical plan, such as by extracting landmarks of the bones in the images and registering the landmarks against a generic three-dimensional model of the joint. In this sense, processor 106 scales the generic three-dimensional model to fit to the X-ray images of the patent to make the three dimensional model patient specific. The surgeon can then identify where the implants should be located and at what pose. This chosen implant configuration is then also part of the surgical plan. As such, the surgical plan comprises three coordinates of the implant (x, y, z) and three pose angles. These coordinates and pose angles are stored in the surgical plan for each implant component. The coordinates and pose angles may be relative to a global reference frame, such as the table of the operating theatre, or relative to the anatomy of the patient, such as a specific bone.

As the operation proceeds, the surgeon uses a surgical instrument 107 for medullary canal preparation with the aim of implementing the surgical plan as closely as possible. However, the surgeon traditionally relies on his own impression of locations and orientations of the surgical instrument 107, which often leads to inaccuracies. Therefore, there is video camera 105 that captures image data of the surgical instrument and processor 106 determines 304 a pose of the surgical instrument 107 relative to the bone or the joint based on the image data from the video camera.

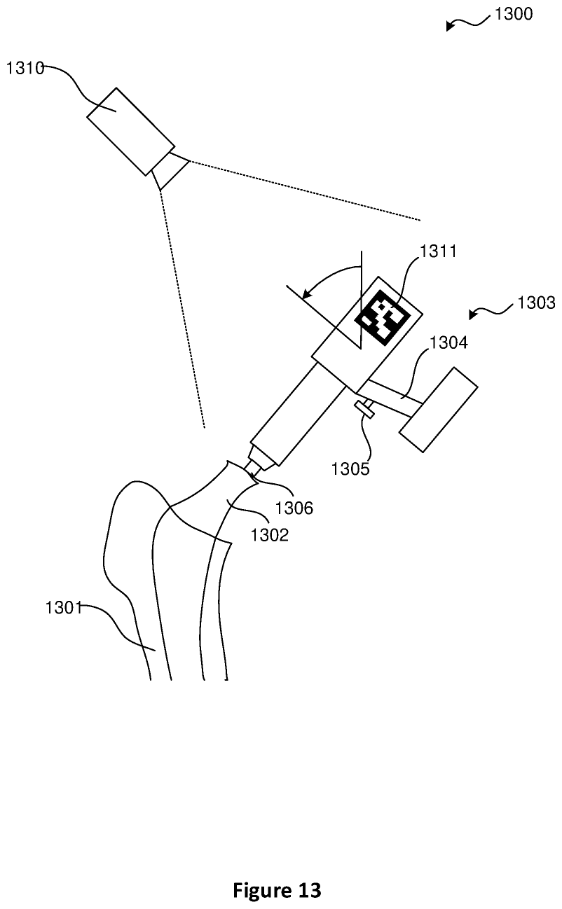

FIG. 13 illustrates an example scenario 1300 of medullary canal preparation of a femur 1301. The surgeon inserts a broach 1302 (or a rasp) into the opening of the canal and either uses a manual impactor, such as a hammer or mallet it apply impact energy to a broach handle, or attaches an impactor 1303 to the broach 1302. In this sense, the impactor 1303 is also considered a surgical instrument. The impactor 1303 has a handle 1304 and a trigger 1305. The surgeon holds the impactor 1303 at handle 1304 and presses the trigger 1305, which activates the impactor 1303. In response the impactor delivers one or more controlled impulses with known energy to the broach 1302, which drives the broach 1302 into the bone 1301.

The impactor 1303 is attached to the broach 1302 by a rigid coupling 1036, such that the pose of the impactor 1303 defines the pose of the broach 1302. In other words, processor 106 has available a fixed spatial relationship between the broach 1302 and the impactor 1303, such as three offset angles and three offset coordinates.

Video camera 1310 (corresponding to video camera 105 and 205 in FIGS. 1 and 2) captures image data of the impactor 1303 and processor 106 determines a pose of the impactor 1303 relative to the bone 1301 or the joint (not shown). More particularly, processor 106 receives the image data from video camera 1310 and detects object features from the image data. The video camera 1310 provides a stream of images contained in the image data at a fixed or variable frame rate, such as 10 fps, 25 fbs, 60 fps or other values. Processor 106 may perform the calculations disclosed herein on each frame individually or may track objects across multiple frames to improve performance. Further, the ultimate information or indication that is provided to the surgeon as a result of the disclosed process may be updated at the same rate as the camera provides the images (the frame rate), which is then referred to a “real-time”.

Processor 106 may have stored an object model of the impactor 1303, such as any combination of shape, size and colour, and attempts to match the object model against objects identified in the image. Once the impactor object model fits to an object in the image, processor 106 can determine the position and pose of the impactor. For example, processor 106 can rotate and shift the impactor object model to optimise the fit and the result is the pose and position of the impactor. The camera may be fixed in relation to the operating table with a known viewing angle and distance, so that processor 106 can calculate the position and pose of impactor 1303 in relation to this global reference frame. Processor 106 may implement feature detection using Haar-like features is disclosed in Viola and Jones, “Rapid object detection using a boosted cascade of simple features”, Computer cool Vision and Pattern Recognition, 2001, which is incorporated herein by reference.

In another example, the image data from camera 1310 also comprises image data of the bone 1301 and processor 106 detects the bone in the image data, such as by, again, fitting a bone object model against objects in the image. Once the bone and the impactor 1303 are identified in the image, the processor 106 can determine the relative position and pose of the impactor 1303 in relation to the bone 1301, such as three offset angles and three translation values.

In yet a further example, there is a geometrical, visible, two-dimensional code 1311 affixed to impactor 1303. This may be an Aruco code and processor 106 may execute an Aruco library available at https://docs.opencv.org/trunk/d9/d6a/group_aruco.html. This enables processor 106 to identify the location and pose of the impactor 1303 without object detection, which may make the process more robust. There may be multiple codes affixed to impactor 1303 to further improve the pose estimation. Again, the pose and position of impactor 1303 may be in relation to bone 1301 or in relation to the joint. In another example, a further marker, such as a further Aruco code is attached to the bone at a predefined landmark to support the detection of the bone 1301 in the image data.

Processor then assesses 306 the pose of the impactor 1303 against the surgical plan. For example, processor 106 assesses whether the current pose, which directly relates to the direction at which the broach 1302 is inserted into the bone 1301, will lead to the planned position and pose of the implant (e.g. the femoral stem 408). In this sense, processor 106 may use a fixed relationship between the pose of impactor 1303 and the final pose of the implant. For example, the pose of the impactor 1303 may be identical to the final pose of the implant if a linear motion of the broach 1302 into the bone 1301 is assumed. In other cases, processor 106 calculates a prediction of the final implant pose based on the current pose of the impactor 1303. For example, processor 106 may use a typical trajectory, which may be available through machine learning of multiple uses of the impactor 1303 to determine a relationship between the current pose of the impactor 1303 and a predicted pose of the implant. It is noted that the broach 1302 is typically different to the actual implant. However, the broach 1302 defines the void into which the implant is later inserted. Therefore, knowing the pose of the broach 1302 also means knowing the pose of the implant.

Processor 106 the provides 308 an indication to the surgeon of a clinical consequence of the pose in relation to the surgical plan. For example, processor 106 may provide an indication of a difference between the planned pose of the implant and the estimated pose of the implant. In a further example, processor 106 may update the surgical plan to reflect the pose of the implant. For example, during preparation of the femur, processor 106 estimates the pose of the femoral component and updates the pose of the acetabular cup to ensure a good correspondence between the two components. In another example, processor 106 performs a kinematic simulation of the anatomical model as disclosed herein, and provides an indication of a performance metric. That metric may be a range of movement, or a risk stratification, such as an indication of a risk of dislocation, impingement or edge loading.

The processor 106 may provide this indication in real-time. This way, the surgeon can see on a computer monitor, for example, how the indication changes as the surgeon rotates the impactor 1303 into a different pose. The surgeon may even rotate the impactor slightly before pressing the trigger button 1305 for the first time. This way, the surgeon can adjust the pose of the impactor 1303 before commencing medullary canal preparation. While the surgeon adjusts the pose of the impactor 1303, the surgeon can see in real-time, how the indication changes. For example, the surgeon can adjust the pose of the impactor until the various risk factors are below an acceptable level. In other words, the surgeon can ‘watch’ the risk levels change as the surgeon moves the impactor 1303 and then press the trigger button 1305 when the risk levels are acceptable.

In some embodiments, the video camera 105 captures the image data of the surgical instrument 107. In particular, the video camera 105 captures the image data of the surgical instrument 107 during the total joint replacement surgery.

The computing device 102 receives the image data of the surgical instrument 107. The processor 106 stores the image data.

As previously mentioned, the computing device 102 processes the image data. In particular, the processor 106 may process the image data. Processing the image data may comprise determining one or more image data parameters. The one or more image data parameters may comprise locations of one or more image data landmarks. The image data landmarks may be, in the case of a total hip replacement, the patient's greater trochanter 1103, lesser trochanter 1105, femoral stem alignment, femoral shaft alignment and/or the centre of rotation of the implanted femoral head 1107. The image data landmarks may alternatively be a feature of, or associated with the surgical instrument 107. The image data landmarks may comprise a number of pelvic landmarks, for example, the anterior superior iliac spine, anterior inferior iliac spine, pubic symphysis, obturator foramen, acetabular floor, sacrum, coccyx and/or greater sciatic notch. The image data landmarks may comprise a number of femoral landmarks, for example, the piriformis fossa and/or intertrochanteric ridge. Each image data landmark may have a determined image data landmark location. The image data landmark location may be a Cartesian coordinate in the reference frame of the image data.

The image data parameters may comprise one or more image data measurements. The image data measurements are indicative of a distance between two or more image data landmarks. The image data measurements may be, for example, a surgical instrument measurement (edge length etc.), leg length, acetabular inclination, acetabular anteversion and/or cement mantle thickness, femoral offset, anterior offset, stem varus/valgus angle, and/or the distance between one or more of the landmarks previously described.

One or more of the image data parameters may correspond with one or more of the digital three-dimensional model parameters. Therefore, one or more of the image data landmarks may correspond with a respective three-dimensional model landmark. Furthermore, one or more of the image data measurements may correspond with a respective three-dimensional model measurement.

In some examples, processing the image data may comprise scaling the image data. The image data may be scaled using a reference object of known dimension that is present in the image data. In some examples, the reference object may be separate from the implant component assembly 405. That is, the reference object may be unrelated to the implant component assembly 405.

In some examples, the image data may be scaled based on a comparison between one or more of the image data parameters and one or more of the three-dimensional model parameters. In said examples, the image data is scaled such that the relevant image data parameter corresponds with the respective three-dimensional model parameters. Alternatively, the magnification can be calculated based on the distance between the observed object (e.g. the joint) and the video camera 105. For example, where the distance between the joint and the video camera 105 is known, the magnification of the image data can be determined.

In some examples, processing the image data comprises detecting one or more edges in the image data. For example, the computing device 102 detects the edges of the surgical instrument 107. The computing device 102 may detect the edges using a suitable edge detection method, such as using a Sobel operator.

In some examples, processing the image data comprises detecting one or more objects in the image data. For example, an anatomical features, e.g. a bone, may be detected in the image data. Furthermore, one or more of the implant component 406 and/or the supplemental implant components 407 may be detected in the image data. In particular, the implant component 406 may be detected in the image data. Alternatively, the surgical instrument 107 may be detected in the image data.

In some examples, the computing device 102 detects the objects in the image data. The computing device 102 may use the detected edges to detect the objects. Alternatively, the computing device 102 may use other features of the image data to detect the objects. The computing device 102 may detect the objects using a suitable object detection method. For example the computing device 102 may use a machine learning method to detect the objects. In some examples, the computing device 102 detects features using the Viola-Jones object detection framework based on Haar features, a scale-invariant feature transform or a histogram of oriented gradients, and uses a classification technique such as a support vector machine to classify the objects.

In some examples, the computing device 102 is also configured to determine the pose of the objects in the image data. For example, after detecting the surgical instrument 107, the computing device 102 is configured to determine the pose of the surgical instrument 107. The pose of the surgical instrument 107 may comprise an indication of the location and orientation of the surgical instrument 107.

In some examples, the computing device 102 may be configured to use the detected edges, objects and/or poses of said objects to determine the one or more image data parameters.

In some examples, the computing device 102 determines one or more differences between the pose of the implant component 406 that will result from the determined pose of the surgical instrument 107 as represented in the image data, and the desired pose of the implant component 406 of the digital three-dimensional model 1200. This is possible because the pose of the implant component 406 is associated with the pose of the surgical instrument 107 as previously described. That is, the pose of the implant component 406 is determined by the pose of the surgical instrument 107.

In some examples, the computing device 102 compares one or more of the image data parameters to one or more parameter thresholds. The parameter thresholds can be indicative of the desired surgical parameters, or acceptable surgical parameters. For example, in the case of the total hip replacement, a parameter threshold can be femoral stem angle threshold. The femoral stem angle of the implanted implant component 406 can be determined from the image data using the determined pose of the surgical instrument 107 as previously described, and this can be compared to the femoral stem angle threshold. In some examples, parameter threshold is a range. The surgeon may specify the parameter thresholds, which may be selected to maximise the postoperative performance of the joint. Alternatively, the computing device 102 can automatically determine the parameter thresholds. If the surgical instrument 107 is determined to deviate from its corresponding parameter thresholds, it can be classified as high risk.

In some examples, the parameter thresholds are equal to the desired surgical parameters. In other examples, the parameter thresholds are threshold ranges centred upon, or including the desired surgical parameter.

The computing device 102 may determine an updated digital three-dimensional model. The computing device 102 updates the pose of the implant component 406 in the digital three-dimensional model 1200 to reflect the pose that the implant component 406 will be implanted in as a result of the surgical instrument pose determined from the image data. As the pose of the surgical instrument 107 is associated with the pose of the implant component 406, the determined surgical instrument pose is used to reflect the actual pose of the implant component 406. Thus, the digital three-dimensional model 1200 is intraoperatively updated to reflect the state the surgery will be in when the implant component 406 is implanted. Updating the pose of the implant component 406 may comprise, for example, translating and/or rotating the implant component 406 of the digital three-dimensional model 1200. The computing device 102 updates the digital three-dimensional model 1200 based on the determined placement of the surgical instrument 107 (and thus, the implant component 406) in the image data in relation to the digital three-dimensional model 1200, thereby determining an updated digital three-dimensional model.

The computing device 102 determines an intraoperative simulated performance metric by simulating movement of the digital three-dimensional model based on the placement of the surgical instrument 107 in the image data. In particular, the assessment module 114 determines the intraoperative simulated performance metric by simulating movement of the updated digital three-dimensional model.

The computing device 102 determines the intraoperative simulated performance metric by performing a kinematic analysis on the updated digital three-dimensional model. The kinematic analysis can comprise moving the relevant portions of the updated digital three-dimensional model to determine a postoperative range of motion of the joint. This movement is performed by the computing device 102 and comprises moving elements of the digital three-dimensional model 1200, such as moving bones against each other. This movement may be defined by the shape and location of bearing surfaces of joints represented by the updated digital three-dimensional model.

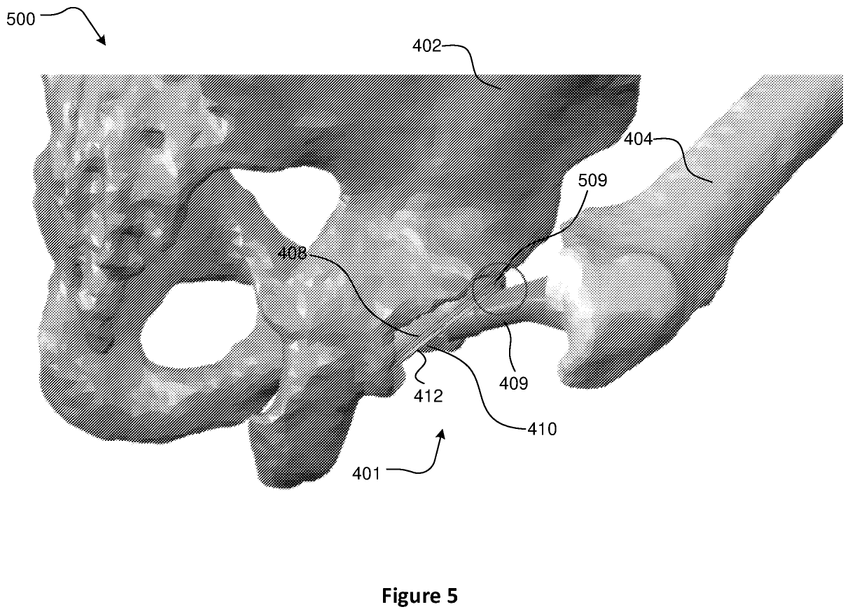

FIG. 5 illustrates an example updated digital three-dimensional model 500 that has been manipulated to determine a postoperative range of motion of a hip joint 501. In particular, updated three-dimensional model 500 of FIG. 5 has been manipulated to simulate seated flexion of the hip joint 501. Included in the updated three-dimensional model 500 is the pelvis 402, femur 404, femoral stem 406, neck 409, implant femoral head 410, liner 412 and acetabular component 408. In the example illustrated in FIG. 5, each surface of the updated digital three-dimensional model 500 is considered solid, and thus a surface of one component contacting another is indicative of a maximum range of motion in that direction. Such an impingement 509 is illustrated in FIG. 5.

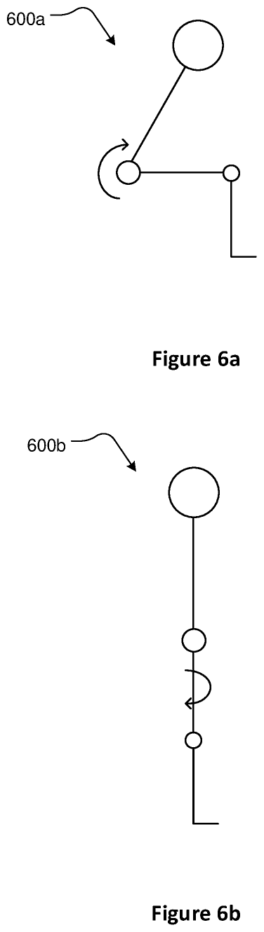

The kinematic analysis may comprise a number of postoperative joint movements. Each postoperative joint movement can simulate a typical movement of the patient after the surgery. For example, in the case of the total hip replacement, the kinematic analysis may comprise the seated flexion movement as shown in FIG. 5. FIG. 6a illustrates a schematic line drawing 600a of a patient performing a seated flexion movement. This movement occurs when the patient rotates their upper body (and thus their pelvis) forward with respect to their femoral head when in a sitting position. This movement also occurs when the patient is in the sitting position and brings their knee upwards towards their torso.

Also in the case of the total hip replacement, the kinematic analysis can comprise a standing pivot extension movement. FIG. 6b illustrates a schematic line drawing 600b of a patient performing a standing pivot extension movement. This movement occurs when the patient is standing and rotates their leg outwards about its longitudinal axis.

The kinematic analysis is associated with at least one kinematic analysis target parameter. Each kinematic analysis target parameter can be indicative of a desired or target performance of the joint. For example, the kinematic analysis target parameter can be an angle representing a target rotation desired of the joint before an impingement occurs. The computing device 102 is configured to provide a risk stratification based on a comparison between the kinematic performance of the updated digital three-dimensional model and the at least one kinematic analysis target parameter.

In some examples, a flexion target parameter can be associated with the seated flexion movement of the kinematic analysis. The flexion target parameter is indicative of a maximum flexion angle achievable by the updated digital three-dimensional model. Furthermore, an extension rotation target parameter can be associated with the standing pivot extension of the kinematic analysis. The extension rotation target is indicative of a maximum rotation angle that the femur can be rotated about the relevant leg's longitudinal axis achievable by the updated digital three-dimensional model.

The computing device 102 may also compare a current (i.e. intraoperative) implant component pose with a number of alternative poses (e.g. of the acetabular component) by determining an alternative simulated performance metric associated with an alternative implant component pose. In other words, the computing device 102 may compare an intraoperative implant component pose with the number of alternative poses. The computing device 102 can adjust the pose of the implant component 406 in the updated digital three-dimensional model, and re-run the kinematic analysis. The computing device 102 is configured to provide an alternative risk stratification based on a comparison between the kinematic performance of the updated digital three-dimensional model with the alternative implant component pose and the at least one kinematic analysis target parameter. For example, the computing device 102 can change the acetabular inclination angle of the acetabular component 408, and re-run the kinematic analysis. In some examples, this can be used to assist the surgeon in determining whether or not the implant component 406 that will implanted should be implanted in a different position.

In some examples, the computing device 102 also determines the alternative simulated performance metric associated with an alternative supplemental implant component 407′. As previously described, the updated digital three-dimensional model includes one or more supplemental implant components 407 that are to be implanted after the implant component 406. The positioning of the implant component 406, which is dictated by the current positioning of the surgical instrument 107, may however mean the originally planned supplemental implant components 407 are unsuitable. Thus, the computing device 102 determines the alternative simulated performance metric associated with the alternative supplemental implant component 407′. The alternative simulated performance metric can be compared to the intraoperative simulated performance metric to assess surgical options. In some examples, this can be used to assist the surgeon in intraoperatively determining appropriate sizing for the supplemental implant components 407.

The computing device 102 determines the alternative supplemental implant component 407′. The computing device 102 can substitute the alternative supplemental implant component 407′ for the supplemental implant component 407 in the updated digital three-dimensional model, and re-run the kinematic analysis. The computing device 102 is configured to provide an alternative risk stratification based on a comparison between the kinematic performance of the updated digital three-dimensional model with the supplemental implant component 407 and the alternative supplemental implant component 407′ using the kinematic analysis target parameter.

In some examples, the computing device 102 determines a preoperative simulated performance metric. The computing device 102 determines the preoperative simulated performance metric by simulating movement of the digital three-dimensional model 1200 according to the surgical plan. In some embodiments, the surgical plan is the digital three-dimensional model 1200. In some embodiments, the surgical plan comprises the digital three-dimensional model 1200, in addition to supplemental information. The surgical plan (and/or the digital three-dimensional model) may comprise a planned placement of the implant component 406 in the digital three-dimensional model 1200.

The computing device 102 determines the preoperative simulated performance metric by performing a preoperative kinematic analysis on the digital three-dimensional model 1200 as previously described with reference to the updated digital three-dimensional model. The preoperative kinematic analysis can comprise moving the relevant portions of the digital three-dimensional model 1200 to determine the surgical plan representing the postoperative range of motion of the joint. This movement is performed by the computing device 102 and comprises moving elements of the digital three-dimensional model 1200, such as moving bones against each other. This movement may be defined by the shape and location of bearing surfaces of joints represented by the digital three-dimensional model 1200. As previously described with reference to the kinematic analysis, the preoperative kinematic analysis may comprise a number of postoperative joint movements. Furthermore, the preoperative kinematic analysis may be associated with at least one preoperative kinematic analysis target parameter. The preoperative kinematic analysis target parameter may correspond with a respective kinematic analysis target parameter associated with the updated digital three-dimensional model.

The computing device 102 may compare the preoperative kinematic analysis with the kinematic analysis. That is, the computing device 102 may compare the preoperative kinematic analysis performed with respect to the digital three-dimensional model 1200 to the kinematic analysis performed with respect to the updated digital three-dimensional model. In some embodiments, the computing device 102 compares the at least one preoperative kinematic analysis target parameter with the corresponding kinematic analysis target parameter. The comparison may be used to, for example update the updated digital three-dimensional model. That is, the computing device 102 may update the updated digital three-dimensional model based on the comparison. For example, one or more of the supplemental implant components 407 may be updated based on the comparison. The update may comprise replacing the existing supplemental implant component 407 of the updated digital three-dimensional model with a different supplemental implant component 407 (e.g. of a different size, manufacturer, material and/or type), and/or may comprise updating the pose of the relevant supplemental implant component 407.

Each implant component 406 and supplemental implant component 407 size comprises unique dimensions and geometry. The progression of implant component 406 and supplemental implant component 407 dimensions are known. Memory 108 can store features of each size of the implant component 406 and/or the supplemental implant components 407. The computing device 102 can compare one or more of the digital three-dimensional model parameters to the features of the each size of the implant component 406 and/or the supplemental implant components 407 and use the comparison to determine an optimized size of the implant component 406, each supplemental implant component 407 and/or the implant component assembly 405.

Each implant component 406 and supplemental implant component 407 size comprises unique dimensions and geometry stored as features in memory 108. The computing device 102 can compare one or more of the digital three-dimensional model parameters to the features of the each size of the implant component 406 and/or the supplemental implant components 407 and use the comparison to determine an optimized pose of the implant component 406, each supplemental implant component 407 and/or the implant component assembly 405.

Furthermore, the computing device 102 can compare one or more of the three-dimensional model parameters to the features of the each size of the implant component 406 and/or the subsequent implant components 407 and use the comparison to determine an optimized pose of each subsequent implant component 407 and/or the implant component assembly 405 in the updated digital three-dimensional model. The computing device 102 can therefore update the updated digital three-dimensional model with an optimised implant component 406 and/or an optimised supplementary implant component(s) 407. This comparison may be based on the risk stratification. This comparison may be based on the determined pose of the surgical instrument 107. The computing device 102 can update the pose of the implant component 406 and/or an supplementary implant component(s) 407 based on this optimisation in the updated digital three-dimensional model. The optimisation may performed with reference to the surgical parameters and/or the parameter thresholds.

At 310, the computing device 102 provides an indication of the intraoperative simulated performance metric as an assessment of a placement of the implant component 406. In particular, the computing device provides the indication of the intraoperative simulated performance metric as an assessment of a current (i.e. intraoperative) placement of the surgical instrument 107. The current placement of the surgical instrument 107 corresponds to a placement of the implant component 406. In providing the indication, the computing device 102 generates the indication of the intraoperative simulated performance metric. In particular, the indication module 116 generates the indication of the intraoperative simulated performance metric. The indication of the intraoperative simulated performance metric is determined as an assessment of the current placement of the surgical instrument 407. The indication of the intraoperative simulated performance metric may also comprise an indication of the one or more alternative simulated performance metrics.

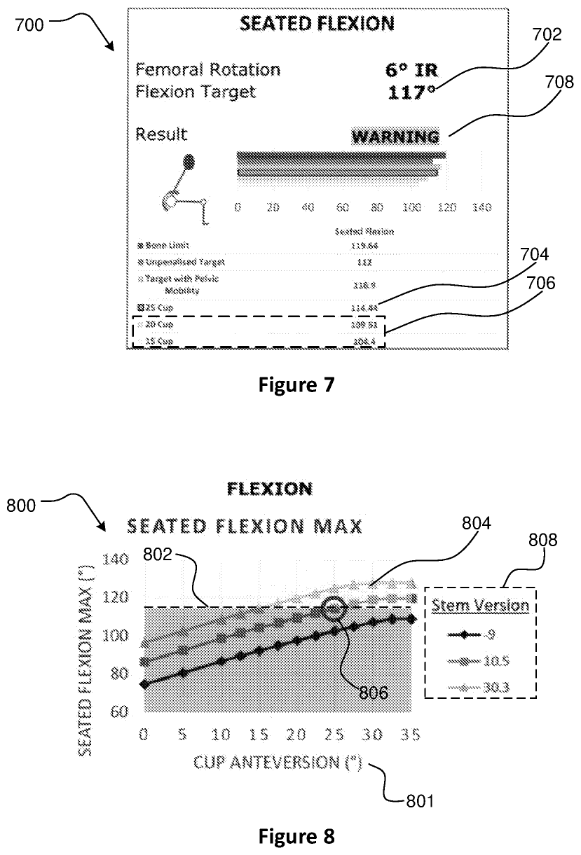

FIG. 7 illustrates an example indication 700 of the intraoperative simulated performance metric determined as an assessment of the current placement of the surgical instrument 107. The indication 700 is associated with the seated flexion kinematic analysis as previously described. The indication 700 includes a intraoperative simulated performance metric 704. The intraoperative simulated performance metric 704 was determined in the kinematic analysis as previously described, and thus is an assessment of the placement of the surgical instrument 107 based on the determined placement of the surgical instrument 107. The indication 700 also includes a plurality of alternative simulated performance metrics 706. The indication 700 includes a kinematic analysis target parameter 702. The indication 700 includes a risk stratification 708. Thus, the indication 700 of the intraoperative simulated performance metric may be considered a risk stratification. In some examples, the risk stratification is indicative of a risk associated with multiple predicted postoperative movements by the patient. The risk stratification may be indicative of a risk of one or more of dislocation of the joint, edge loading, and postoperative joint pain. Providing the indication 700 to the surgeon may comprise displaying a graphic similar to that in FIG. 7 to the surgeon on a computer screen, or may comprise printing the graphic. The indication may also take other forms, such as a numerical score only, a bar chart as in FIG. 7, a traffic light scale (red, yellow, or green). The indication may also be audio (beep, generated voice, natural language generation) or other indicators like vibrations etc.

FIG. 8 illustrates an alternative indication 800 of the intraoperative simulated performance metric determined as an assessment of placement of the surgical instrument 107. The indication 800 is generated in accordance with the kinematic analysis based on the updated digital three-dimensional model, and a number of alternative kinematic analyses based on alternative implant component poses, and alternative supplemental implant component sizes. The indication 800 includes a kinematic analysis target parameter 802.

The indication 800 includes a plurality of simulated performance metrics 804. The simulated performance metrics 804 may comprise at least one intraoperative simulated performance metric. The simulated performance metrics 804 were determined in the kinematic analyses previously described, and thus are an assessment of the placement of the surgical instrument 107 and selection of the supplemental implant components 407 based on the determined placement of the surgical instrument 107. Each simulated performance metric 804 (plotted against the y-axis) is a maximum seated flexion angle. Each simulated performance metric 804 is associated with a corresponding implant component parameter 801 (the x-axis). Each simulated performance metric 804 corresponds with a respective kinematic analysis performed with the particular implant component parameter 801 and supplemental implant component parameter 808. The circled simulated performance metric 806 corresponds with the kinematic analysis performed with respect to the updated digital three-dimensional model. That is, the circled simulated performance metric 806 can be considered the intraoperative simulated performance metric. Simulated performance metrics 804 above the kinematic analysis target parameter 802 represent low risk options. That is, the surgery being completed with parameters as per the simulated performance metrics 804 above the kinematic analysis target parameter 802 are less likely to result in a problematic outcome than the surgery being completed with parameters as per the simulated performance metrics 804 below the kinematic analysis target parameter 802. Thus, the indication 800 of the simulated performance metrics may be considered a risk stratification. In some examples, the risk stratification is indicative of a risk associated with multiple predicted postoperative movements by the patient. The risk stratification may be indicative of a risk of one or more of dislocation of the joint, edge loading, and postoperative joint pain. In some examples, the indication 700 and the indication 800 may be presented together as the indication of the intraoperative simulated performance metric.

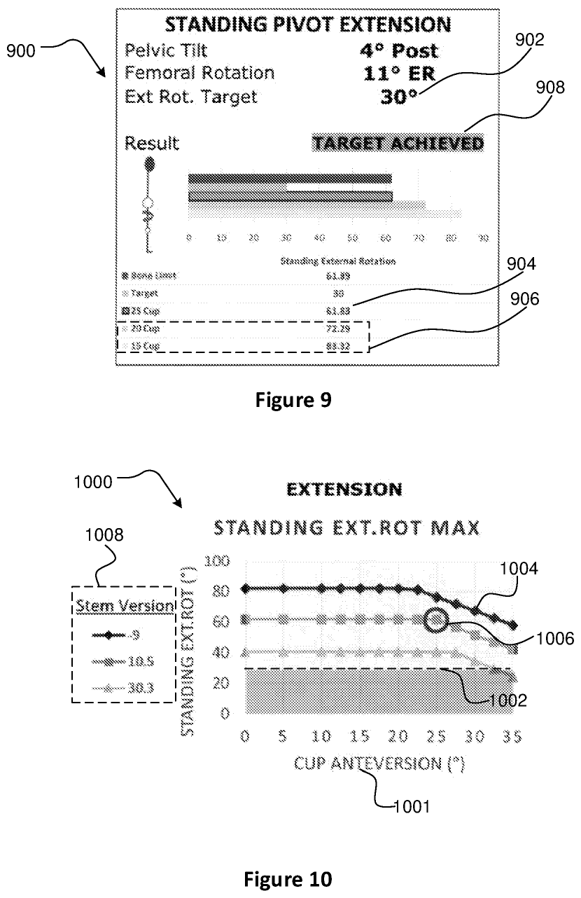

FIG. 9 illustrates another example indication 900 of the intraoperative simulated performance metric as an assessment of a placement of the implant component 406. The indication 900 is associated with the standing pivot extension kinematic analysis as previously described. The indication 900 includes an intraoperative simulated performance metric 904. The intraoperative simulated performance metric 904 was determined in the kinematic analysis previously described, and thus is an assessment of the placement of the surgical instrument 107. The indication 900 also includes a plurality of alternative simulated performance metrics 906. The indication 900 also includes an example kinematic analysis target parameter 902. The indication 900 includes a risk stratification 908. Thus, the indication 900 of the intraoperative simulated performance metric may be considered a risk stratification. In some examples, the risk stratification is indicative of a risk associated with multiple predicted postoperative movements by the patient. The risk stratification may be indicative of a risk of one or more of dislocation of the joint, edge loading, and postoperative joint pain.

FIG. 10 illustrates an alternative indication 1000 of the simulated performance metric determined as an assessment of the placement of the surgical instrument 107. The indication 1000 is generated in accordance with the kinematic analysis based on the updated digital three-dimensional model, and a number of alternative kinematic analyses based on alternative implant component poses, and alternative supplemental implant component 407′ sizes. The indication 1000 includes a kinematic analysis target parameter 1002.

The indication 1000 includes a plurality of simulated performance metrics 1004. The simulated performance metrics 1004 were determined in the kinematic analyses previously described, and thus are an assessment of the placement of the surgical instrument 107 and selection of the supplemental implant components 407. Each simulated performance metric 1004 (plotted against the y-axis) is a maximum standing pivot extension angle as previously described. Each simulated performance metric 1004 is associated with a corresponding implant component parameter 1001 (the x-axis). Each simulated performance metric 1004 corresponds with a respective kinematic analysis performed with the particular implant component parameter 1001 and supplemental implant component parameter 1008. The circled simulated performance metric 1006 corresponds with the kinematic analysis performed with respect to the updated digital three-dimensional model. That is, the circled simulated performance metric 1006 can be considered the intraoperative simulated performance metric.

Simulated performance metrics 1004 above the kinematic analysis target parameter 1002 represents low risk options. That is, the surgery being completed with parameters as per the simulated performance metrics 1004 above the kinematic analysis target parameter 1002 are less likely to result in a problematic outcome than the surgery being completed with parameters as per the simulated performance metrics 1004 below the kinematic analysis target parameter 1002. Thus, the indication 1000 of the simulated performance metrics may be considered a risk stratification. In some examples, the risk stratification is indicative of a risk associated with multiple predicted postoperative movements by the patient. The risk stratification may be indicative of a risk of one or more of dislocation of the joint, edge loading, and postoperative joint pain. In some examples, the indication 9000 and the indication 1000 may be presented together as the indication of the intraoperative simulated performance metric.

The processor 106 is configured to encode the indication of the intraoperative simulated performance metric into one or more display object(s). The display object can be in the form of a bitmap (e.g. a PNG or JPEG file) that illustrates the indication of the intraoperative simulated performance metric. Alternatively, the display object can be in the form of intraoperative simulated performance metric indication display program code executable to cause display of the indication.

The computing device 102 provides the indication of the intraoperative simulated performance metric as the assessment of the current placement of the surgical instrument 107. In particular, the visualisation module 118 is configured to provide the indication of the intraoperative simulated performance metric. The computing device 102 displays the indication using the user interface 120. In some examples, the computing device 102 is configured to execute the performance metric indication display program code, thereby rendering the encoded indication of the intraoperative simulated performance metric on the user interface 120.

### Method 300 Performed by System 200

In some examples, the method 300, or part thereof, can be performed by a remote computing device. For example, as described below, 302, 304, 306 and 308 may be performed by the information processing device 203 that is remote from the computing device 202, the video camera 205 and/or the imaging device 204. This can be advantageous where the computational specification(s) of the computing device 202 is insufficient to perform one or more of the steps of the method 300.

In some examples, the information processing device 203 generates the digital three-dimensional model 1200. In some examples, another computing device generates the digital three-dimensional model 1200. The digital three-dimensional model 1200 is a digital model. The digital three-dimensional model 1200 may be a hip, knee, shoulder, elbow or another joint. The digital three-dimensional model 1200 can comprise an anatomical three-dimensional model 1202 and an implant component assembly three-dimensional model 1204, and can be generated as previously described with reference to method 300 being performed by system 100. That is, the digital three-dimensional model 1200 is generated using information provided by the preoperative imaging device, and is a three-dimensional model of the joint to be replaced in the joint replacement surgery. Furthermore, as previously described, the implant component assembly three-dimensional model 1204 is a three-dimensional representation of the implant component assembly 405.

In some examples, the preoperative imaging device may be configured to provide the information to the processor 206. The processor 206 processes the information provided by the preoperative imaging device to generate the anatomical three-dimensional model 1202. The anatomical three-dimensional model 1202 can be stored in memory 208.

In some examples, a model generating computing device (not shown) processes the information provided by the preoperative imaging device to generate the anatomical three-dimensional model 1202. In said examples, the anatomical three-dimensional model 1202 can be provided to the information processing device 203. The anatomical three-dimensional model 1202 can be stored in memory 208.

The digital three-dimensional model 1200 represents the intended joint configuration after the surgery by comprising the anatomical three-dimensional model 1202 and the implant component assembly three-dimensional model 1204.

In some examples the information processing device 203 processes the digital three-dimensional model 1200. In such cases, the digital three-dimensional model 1200 may be processed by the processor 106. In some examples, the model generating computing device or another computing device processes the digital three-dimensional model 1200 and transmits the processed digital three-dimensional model 1200 to the information processing device 203. Processing the digital three-dimensional model 1200 may comprise determining one or more digital three-dimensional model parameters, landmarks and/or measurements as previously described.

The information processing device 203 stores the surgical plan in memory 108. That is, the information processing device 203 stores the digital three-dimensional model 1200 in memory 208. The information processing device 203 stores the digital three-dimensional model 1200, and the associated digital three-dimensional model parameters.

Returning to FIG. 3, processor 206 stores 302 the surgical plan. The surgical plan comprises information about the planned position (location and pose) of the one or more implant components that are to be implanted into the patient to replace the joint. As previously described, the surgical plan is created preoperatively and may be based on medical imaging data, such as preoperative X-ray images, CT scans or others.

As the operation proceeds, the surgeon uses a surgical instrument 207 for medullary canal preparation with the aim of implementing the surgical plan as closely as possible. However, the surgeon traditionally relies on his own impression of locations and orientations of the surgical instrument 207, which often leads to inaccuracies. Therefore, there is video camera 205 that captures image data of the surgical instrument and processor 106 determines 304 a pose of the surgical instrument 207 relative to the bone or the joint based on the image data from the video camera.

As previously described, FIG. 13 illustrates an example scenario 1300 of medullary canal preparation of a femur 1301.

The impactor 1303 is attached to the broach 1302 by a rigid coupling 1036, such that the pose of the impactor 1303 defines the pose of the broach 1302. In other words, processor 106 has available a fixed spatial relationship between the broach 1302 and the impactor 1303, such as three offset angles and three offset coordinates.

Video camera 1310 (corresponding to video camera 105 and 205 in FIGS. 1 and 2) captures image data of the impactor 1303 and processor 206 determines a pose of the impactor 1303 relative to the bone 1301 or the joint (not shown). More particularly, processor 206 receives the image data from video camera 1310 and detects object features from the image data. The ultimate information or indication that is provided to the surgeon as a result of the disclosed process may be updated at the same rate as the camera provides the images (the frame rate), which is then referred to a “real-time”.

Processor 206 may have stored an object model of the impactor 1303, such as any combination of shape, size and colour, and attempts to match the object model against objects identified in the image. Once the impactor object model fits to an object in the image, processor 206 can determine the position and pose of the impactor as previously described.

In another example, the image data from camera 1310 also comprises image data of the bone 1301 and processor 106 detects the bone in the image data, such as by, again, fitting a bone object model against objects in the image. Once the bone and the impactor 1303 are identified in the image, the processor 206 can determine the relative position and pose of the impactor 1303 in relation to the bone 1301, such as three offset angles and three translation values.

In yet a further example, there is a geometrical, visible, two-dimensional code 1311 affixed to impactor 1303. This may be an Aruco code and processor 206 may execute an Aruco library available at https://docs.opencv.org/trunk/d9/d6a/group_aruco.html. This enables processor 206 to identify the location and pose of the impactor 1303 without object detection, which may make the process more robust. There may be multiple codes affixed to impactor 1303 to further improve the pose estimation. Again, the pose and position of impactor 1303 may be in relation to bone 1301 or in relation to the joint. In another example, a further marker, such as a further Aruco code is attached to the bone at a predefined landmark to support the detection of the bone 1301 in the image data.

Processor then assesses 306 the pose of the impactor 1303 against the surgical plan as previously described.

For example, processor 206 assesses whether the current pose, which directly relates to the direction at which the broach 1302 is inserted into the bone 1301, will lead to the planned position and pose of the implant (e.g. the femoral stem 408). In this sense, processor 206 may use a fixed relationship between the pose of impactor 1303 and the final pose of the implant.

Processor 206 the provides 308 an indication to the surgeon of a clinical consequence of the pose in relation to the surgical plan. For example, processor 206 may provide an indication of a difference between the planned pose of the implant and the estimated pose of the implant. In a further example, processor 206 may update the surgical plan to reflect the pose of the implant. For example, during preparation of the femur, processor 206 estimates the pose of the femoral component and updates the pose of the acetabular cup to ensure a good correspondence between the two components. In another example, processor 206 performs a kinematic simulation of the anatomical model as disclosed herein, and provides an indication of a performance metric. That metric may be a range of movement, or a risk stratification, such as an indication of a risk of dislocation, impingement or edge loading. The processor 206 may provide this indication in real-time as previously described.

The video camera 205 captures the image data of the surgical instrument 207. In particular, the video camera 205 captures the image data of the surgical instrument during the total joint replacement surgery.

The information processing device 203 receives image data of the surgical instrument 207. The processor 206 stores the image data of the surgical instrument 207 in memory 208. In some examples, the video camera 205 transmits the image data to the information processing device 203 over the communications network 250.

The information processing device 203 processes the image data. In particular, the processor 206 may process the image data. Processing the image data may comprise determining one or more image data parameters. The one or more image data parameters may comprise locations of one or more image data landmarks as previously described. Each image data landmark may have a determined image data landmark location as previously described. The image data parameters may comprise one or more image data measurements as previously described. For example, the image data measurements are indicative of a distance between two or more image data landmarks.

One or more of the image data parameters may correspond with one or more of the digital three-dimensional model parameters. Therefore, one or more of the image data landmarks may correspond with a respective three-dimensional model landmark. Furthermore, one or more of the image data measurements may correspond with a respective three-dimensional model measurement.

In some examples, processing the image data comprises scaling the image data.

In some examples, processing the image data comprises detecting one or more edges in the image data. For example, the information processing device 203 detects the edges of the surgical instrument 207. The information processing device 203 may detect the edges using a suitable edge detection method, such as using a Sobel operator.

In some examples, processing the image data comprises detecting one or more objects in the image data. For example, an anatomical feature, e.g. a bone, may be detected in the image data. Furthermore, one or more of the implant component 406 and/or the supplemental implant components 407 may be detected in the image data. The surgical instrument 207 may be detected in the image data.

The information processing device 203 detects the objects in the image data.

In some examples, the information processing device 203 also determines the pose of the objects in the image data. For example, after detecting the surgical instrument 207, the information processing device 203 is configured to determine the pose of the surgical instrument 207. The pose of the surgical instrument 207 may comprise an indication of the location and orientation of the surgical instrument 207.

In some examples, the information processing device 203 uses the detected edges, objects and/or poses of said objects to determine the one or more image data parameters.

In some examples, the information processing device 203 determines one or more differences between the pose of the implant component 406 that will result from the determined pose of the surgical instrument 207 as represented in the image data, and the pose of the implant component 406 of the digital three-dimensional model 1200. This is possible because the pose of the implant component 406 is associated with the pose of the surgical instrument 207 as previously described. That is, the pose of the implant component 406 is determined by the pose of the surgical instrument 207.

In some examples, the information processing device 203 compares one or more of the image data parameters to one or more parameter thresholds. The parameter thresholds can be indicative of the desired surgical parameters, or acceptable surgical parameters. For example, in the case of the total hip replacement, a parameter threshold can be a femoral stem angle threshold. The femoral stem angle of the implanted implant component 406 can be determined from the image data as previously described, and this can be compared to the femoral stem angle threshold. In some examples, parameter threshold is a range. The surgeon may specify the parameter thresholds, which may be selected to maximise the postoperative performance of the joint. Alternatively, the information processing device 203 can automatically determine the parameter thresholds. If the surgical instrument 207 is determined to deviate from its corresponding parameter thresholds, it can be classified as high risk.

In some examples, the parameter thresholds are equal to the desired surgical parameters. In other examples, the parameter thresholds are threshold ranges centred upon, or including the desired surgical parameter.

The information processing device 203 may determine an updated digital three-dimensional model. The information processing device 203 updates the pose of the implant component 406 in the digital three-dimensional model 1200 to reflect the pose that the implant component 406 will be implanted in as a result of the surgical instrument pose determined from the image data. As the pose of the surgical instrument 207 is associated with the pose of the implant component 406, the determined surgical instrument pose is used to reflect the actual pose of the implant component 406. Thus, the digital three-dimensional model 1200 is intraoperatively updated to reflect the state the surgery will be in when the implant component 406 is implanted. Updating the pose of the implant component 406 may comprise, for example, translating and/or rotating the implant component 406 of the digital three-dimensional model 1200. The information processing device 203 updates the digital three-dimensional model 1200 based on the determined placement of the surgical instrument 407 in the image data in relation to the digital three-dimensional model 1200, thereby determining an updated digital three-dimensional model.

The information processing device 203 determines an intraoperative simulated performance metric by simulating movement of the updated digital three-dimensional model based on the placement of the surgical instrument 207 in the image data. In particular, the assessment module 114 determines the intraoperative simulated performance metric by simulating movement of the updated digital three-dimensional model.

The information processing device 203 determines the intraoperative simulated performance metric by performing a kinematic analysis on the updated digital three-dimensional model. The kinematic analysis can comprise moving the relevant portions of the updated digital three-dimensional model to determine a postoperative range of motion of the joint. This movement is performed by the information processing device 203 and comprises moving elements of the digital three-dimensional model 1200, such as moving bones against each other. This movement may be defined by the shape and location of bearing surfaces of joints represented by the updated digital three-dimensional model.

The kinematic analysis performed by the information processing device 203 may be as described with reference to system 100 and at least FIGS. 5 and 6. That is, the kinematic analysis may comprise a number of postoperative joint movements. Each postoperative joint movement can simulate a typical movement of the patient after the surgery. The kinematic analysis may comprise the seated flexion movement and/or the standing pivot extension movement as previously described.

As previously described, the kinematic analysis is associated with at least one kinematic analysis target parameter. Each kinematic analysis target parameter can be indicative of a desired or target performance of the joint. For example, the kinematic analysis target parameter can be an angle representing a target rotation desired of the joint before an impingement occurs. The information processing device 203 is configured to provide a risk stratification based on a comparison between the kinematic performance of the updated digital three-dimensional model and the at least one kinematic analysis target parameter.

In some examples, a flexion target parameter can be associated with the seated flexion movement of the kinematic analysis as described with reference to system 100. Furthermore, an extension rotation target parameter can be associated with the standing pivot extension of the kinematic analysis as described with reference to system 100.

The information processing device 203 may also compare a current (i.e. intraoperative) implant component pose with a number of alternative poses (e.g. of the acetabular component) by determining an alternative simulated performance metric associated with an alternative implant component pose. In other words, the information processing device 203 may compare an intraoperative implant component pose with the number of alternative poses. The information processing device 203 can adjust the pose of the implant component 406 in the updated digital three-dimensional model, and re-run the kinematic analysis. The information processing device 203 is configured to provide an alternative risk stratification based on a comparison between the kinematic performance of the updated digital three-dimensional model with the alternative implant component pose and the at least one kinematic analysis target parameter. For example, the information processing device 203 can change the acetabular inclination angle of the acetabular component 408, and re-run the kinematic analysis. In some examples, this can be used to assist the surgeon in determining whether or not the implant component 406 that will be implanted should be implanted in a different position as previously described.

In some examples, the information processing device 203 also determines the alternative simulated performance metric associated with an alternative supplemental implant component 407′. As previously described, the updated digital three-dimensional model includes one or more supplemental implant components 407 that are to be implanted after the implant component 406. The positioning of the implant component 406, which is dictated by the current positioning of the surgical instrument 207, may however mean the originally planned supplemental implant components 407 are unsuitable. Thus, the information processing device 203 determines the alternative simulated performance metric associated with the alternative supplemental implant component 407′. The alternative simulated performance metric can be compared to the intraoperative simulated performance metric to assess surgical options. In some examples, this can be used to assist the surgeon in intraoperatively determining appropriate sizing for the supplemental implant components 407.

The information processing device 203 determines the alternative supplemental implant component 407′. The computing device 102 can substitute the alternative supplemental implant component 407′ for the supplemental implant component 407 in the updated digital three-dimensional model, and re-run the kinematic analysis. The information processing device 203 is configured to provide an alternative risk stratification based on a comparison between the kinematic performance of the updated digital three-dimensional model with the supplemental implant component 407 and the alternative supplemental implant component 407′ using the kinematic analysis target parameter.

In some examples, the information processing device 203 determines a preoperative simulated performance metric. The information processing device 203 determines the preoperative simulated performance metric by simulating movement of the digital three-dimensional model 1200 according to a surgical plan. In some embodiments, the surgical plan is the digital three-dimensional model 1200. In some embodiments, the surgical plan comprises the digital three-dimensional model 1200, in addition to supplemental information. The surgical plan (and/or the digital three-dimensional model) may comprise a planned placement of the implant component in the digital three-dimensional model 1200.

The information processing device 203 determines the preoperative simulated performance metric by performing a preoperative kinematic analysis on the digital three-dimensional model as previously described with reference to the updated digital three-dimensional model. The preoperative kinematic analysis can comprise moving the relevant portions of the digital three-dimensional model 1200 to determine the surgical plan representing the postoperative range of motion of the joint. This movement is performed by the information processing device 203 and comprises moving elements of the digital three-dimensional model, such as moving bones against each other.

The preoperative kinematic analysis may be associated with at least one preoperative kinematic analysis target parameter. The preoperative kinematic analysis target parameter may correspond with a respective kinematic analysis target parameter associated with the updated digital three-dimensional model.

The information processing device 203 may compare the preoperative kinematic analysis with the kinematic analysis. That is, the information processing device 203 may compare the preoperative kinematic analysis performed with respect to the digital three-dimensional model 1200 to the kinematic analysis performed with respect to the updated digital three-dimensional model. In some embodiments, the information processing device 203 compares the at least one preoperative kinematic analysis target parameter with the corresponding kinematic analysis target parameter. The comparison may be used to, for example update the updated digital three-dimensional model. That is, the information processing device 203 may update the updated digital three-dimensional model based on the comparison. For example, one or more of the supplemental implant components 407 may be updated based on the comparison. The update may comprise replacing the existing supplemental implant component 407 of the updated digital three-dimensional model with a different supplemental implant component 407 (e.g. of a different size, manufacturer, material and/or type), and/or may comprise updating the pose of the relevant supplemental implant component 407.

At 310, the information processing device 203 provides an indication of the intraoperative simulated performance metric as an assessment of a placement of the implant component. In particular, the information processing device 203 provides the indication of the intraoperative simulated performance metric as an assessment of a current (i.e. intraoperative) placement of the surgical instrument 207. The current placement of the surgical instrument 207 corresponds to a placement of the implant component 406. In providing the indication, the information processing device 203 generates an indication of the intraoperative simulated performance metric. In particular, the indication module 116 generates the indication of the intraoperative simulated performance metric. The indication of the intraoperative simulated performance metric is determined as an assessment of a placement of the surgical instrument 207. The indication of the intraoperative simulated performance metric may also comprise an indication of the one or more alternative simulated performance metrics.

The information processing device 203 may generate an indication 700 of the intraoperative simulated performance metric as described with reference to system 100 and FIGS. 7, 8, 9 and/or 10.

The processor 206 is configured to encode the indication of the intraoperative simulated performance metric into one or more display object(s). The display object can be in the form of a bitmap (e.g. a PNG or JPEG file) that illustrates the indication of the intraoperative simulated performance metric. Alternatively, the display object can be in the form of intraoperative simulated performance metric indication display program code executable to cause display of the indication. The information processing device 203 is configured to transmit the one or more display objects to the computing device 202 using the communications network 250.

The computing device 202 provides the indication of the intraoperative simulated performance metric as the assessment of a placement of the implant component 406. The computing device 202 is configured to execute the performance metric indication display program code, thereby rendering the encoded indication of the intraoperative simulated performance metric on the user interface 120.

### Advantages

As previously described, surgeons can modify a large number of parameters in surgeries, and in particular, in joint replacement surgeries. The disclosed examples enable the surgeon to intraoperatively assess the progress of the surgery, and continue, or adjust the course of the surgery in accordance with feedback provided by the disclosed examples.

By generating and storing the digital three-dimensional model 1200 of the joint, the surgeon has available a detailed surgical plan that can be used as a target outcome for the surgery. Intraoperatively capturing the image data enables intraoperative analysis of surgical progress.

Incorrectly implanting the implant component 406 can result in a number of undesirable postoperative outcomes. For example, in total hip replacements, incorrect femoral stem positioning can increase the risk of postoperative joint dislocations, edge loading and joint pain. Postoperative joint dislocations cause great discomfort to the patient, and can require subsequent surgical intervention. Edge loading can cause premature wear of the joint. Joint pain again causes discomfort to the patient.

In the disclosed examples, processor 106 updates the digital three-dimensional model 1200 based on the determined pose of the surgical instrument. The pose of the surgical instrument is determined from the image data, and enables simulation and optimisation of the performance of the joint.

The disclosed kinematic analysis is used to determine the intraoperative simulated performance metric of the joint based on the updated digital three-dimensional model. The intraoperative simulated performance metric is provided to the surgeon, and provides the surgeon with an insight into the future performance or the joint during the operation. Where the intraoperative simulated performance metric indicates there is a high risk of an undesirable postoperative outcome, the surgeon may adjust one or more of the surgical parameters accordingly to attempt to improve it. For example, the surgeon may attempt to reposition the surgical instrument. Alternatively, the surgeon may select an alternative implant component 406, or supplemental implant components 407 to compensate for the state of the surgical instrument.

Some examples pre-operatively support the surgeon's decision making process by performing the kinematic analysis across a range of implant component 406 poses, and supplemental implant component 407 sizes. The results of this analysis may be presented to the surgeon in the form of a risk stratification. Furthermore, some examples can determine optimised parameters, and make corresponding suggestions to the surgeon. For example, some examples can suggest optimised supplemental implant component 407 sizes that minimise the risk of postoperative complications.

It will be appreciated by persons skilled in the art that numerous variations and/or modifications may be made to the above-described examples, without departing from the broad general scope of the present disclosure. The present examples are, therefore, to be considered in all respects as illustrative and not restrictive.

It will be appreciated by persons skilled in the art that numerous variations and/or modifications may be made to the specific examples without departing from the scope as defined in the claims.

It should be understood that the techniques of the present disclosure might be implemented using a variety of technologies. For example, the methods described herein may be implemented by a series of computer executable instructions residing on a suitable computer readable medium. Suitable computer readable media may include volatile (e.g. RAM) and/or non-volatile (e.g. .ROM, disk) memory, carrier waves and transmission media. Exemplary carrier waves may take the form of electrical, electromagnetic or optical signals conveying digital data streams along a local network or publically accessible network such as the internet.

It should also be understood that, unless specifically stated otherwise as apparent from the following discussion, it is appreciated that throughout the description, discussions utilizing terms such as “estimating” or “processing” or “computing” or “calculating”, “optimizing” or “determining” or “displaying” or “maximising” or the like, refer to the action and processes of a computer system, or similar electronic computing device, that processes and transforms data represented as physical (electronic) quantities within the computer system's registers and memories into other data similarly represented as physical quantities within the computer system memories or registers or other such information storage, transmission or display devices.

The present examples are, therefore, to be considered in all respects as illustrative and not restrictive.

1. A system for assisting a surgeon in implanting a joint replacement implant component during a surgery of replacing a joint, the system comprising:
an instrument for medullary canal preparation;
a video camera to capture image data of the instrument;
a computer system to:
store a surgical plan;
determine a pose of the instrument relative to the bone or the joint based on the image data from the video camera;
assess the pose of the instrument against the surgical plan; and
provide an indication to the surgeon of a clinical consequence of the pose in relation to the surgical plan.

2. The system of claim 1, wherein the surgical plan comprises a two-dimensional plan.

3. The system of claim 1 or 2, wherein the computer system is configured to create a three-dimensional surgical plan from two or more two-dimensional medical images.

4. The system of claim 3, wherein the medical images are X-ray images.

5. The system of any one of the preceding claims, wherein the surgical plan comprises a three-dimensional surgical plan.

6. The system of any one of the preceding claims, wherein the instrument is one of a broaching instrument and a rasping instrument.

7. The system of any one of the preceding claims, wherein the instrument comprises a broach handle with an impact surface to receive impact from a surgeon operated hammer.

8. The system of any one of the preceding claims, wherein the instrument is an automatic impactor that generates impact energy and delivers the impact energy to a broach for medullary canal preparation.

9. The system of claim 8, wherein the automatic impactor is controlled.

10. The system of claim 8 or 9, wherein the impactor delivers a predefined amount of energy to the broach.

11. The system of any one of the preceding claims, wherein the clinical consequence comprises a risk stratification.

12. The system of any one of the preceding claims, wherein the clinical consequence comprises a simulated performance metric determined by simulating a three-dimensional model of the joint based on the pose of the broaching instrument.

13. The system of claim 12, wherein simulating the three-dimensional model is based on an implant placement defined by the pose of the broaching instrument.

14. The system of any one of the preceding claims, wherein the computer system is configured to generate a graphical display of the joint and an indication of the pose of the broaching instrument in relation to the joint.

15. The system of any one of the preceding claims, wherein the computer system is further configured to:
receive an x-ray image;
display the x-ray image; and
overlay over the x-ray image an indication of the pose of the broaching instrument.

16. The system of any one of the preceding claims, wherein determining the pose of the instrument comprises detecting objects in the image data and fitting an object model to the objects.

17. The system of any one of the preceding claims, wherein a two-dimensional marker is affixed to the instrument and determining the pose of the instrument comprises determining the pose of the two-dimensional marker.

18. A method for assisting a surgeon in implanting a joint replacement implant component during a surgery of replacing a joint, the method comprising:
storing a surgical plan;
determining a pose of the instrument relative to the bone or the joint based on the image data from a video camera;
assessing the pose of the instrument against the surgical plan; and
providing an indication to the surgeon of a clinical consequence of the pose in relation to the surgical plan.

19. The method of claim 18, wherein the clinical consequence comprises a risk stratification.

20. The method of claim 18 or claim 19, wherein the clinical consequence comprises a simulated performance metric determined by simulating a three-dimensional model of the joint based on the pose of the broaching instrument.

21. The method of claim 20, wherein simulating the three-dimensional model is based on an implant placement defined by the pose of the broaching instrument.

22. The method of any one of claims 18 to 21, wherein determining the pose of the instrument comprises detecting objects in the image data and fitting an object model to the objects.

23. The method of any one of claims 18 to 22, wherein a two-dimensional marker is affixed to the instrument and determining the pose of the instrument comprises determining the pose of the two-dimensional marker.
